(19) United States
(12) Patent Application Publication
(10) Pub. No.: US 2022/0328184 A1
(43) Pub. Date: Oct. 13, 2022

(54) Diagnostic and assessment system for mental illness based on collecting and analyzing multifactorial data using machine learning and artificial intelligence algorithms.

(71) Applicant: Fayyaz Memon, Bethesda, MD (US)

(72) Inventor: Fayyaz Memon, Bethesda, MD (US)

(21) Appl. No.: 17/301,619

(22) Filed: Apr. 9, 2021

(51) Int. Cl. G16H 50/20; G16H 50/30; G16H 70/40; G16H 20/70; G16H 50/50

(52) U.S. Cl. CPC G16H 50/20; G16H 50/30; G16H 70/40; G16H 20/70; G16H 50/50

(57) ABSTRACT

This process is based on a multifactorial diagnostic approach, treatment assessment using machine learning, and artificial intelligence algorithms for mental illness. The system consolidates inputs from genetic reports, imaging results, neurological tests, and clinical information. It compares the data from current research to develop a score using machine learning algorithms and data analysis techniques, including linear and logistics regression, decision trees, Naive Bayes, and ensemble methods. The scoring is based on multiple factors, including genetic, imaging, medical, lab, neurological, and clinical interviews. The scoring algorithm for data analysis and overlay methods improves sensitivity and specificity for diagnosing mental disorders. This process and system create a treatment assessment based on the diagnosis and pharmacogenetics, and medical risk factors to refine the targeted treatment plan and reduce the side effects. The system is dynamic and continuously updating diagnosis or treatment based on the new incoming data using statistical modeling techniques.

### CROSS-REFERENCE TO RELATED APPLICATIONS

This application claims the benefit of provisional patent application Ser. No., U.S. 62/834,286 filed on Apr. 15, 2010 by the present inventor.

### FEDERALLY SPONSORED RESEARCH “Not Applicable”

### BACKGROUND

### Prior Art

| Pat. No. | Kind Code | Issue Date | Patentee |
|---|---|---|---|
| 10,478,112 | B2 | Nov. 19, 2019 | Dennis Wall |
| 10,223,640 | B2 | Mar. 5, 2019 | Agueda, Herbert |
| 10,026,508 | B2 | Jul. 17, 2019 | Julio, Dino |
| 10,325,070 | B2 | Jun. 18, 2019 | Ryan Gordon Beale |
| 9,396,486 | B2 | Jul. 19, 2016 | John M. Stivoric |

There has been no reliable diagnostic approach to treat mental illness as it varies highly from person to person and provider to provider. These treatments are generally based upon one-one meetings with a therapist or psychiatrist or a combination of both psychotherapy and psychiatric medication. Psychotherapy may include evidence based-techniques such as CBT and DBT. The other approaches may include meditation, light therapy, yoga, etc. Therapist are trained psychologist who generally start with psychotherapy and other evidence techniques whereas psychiatrist generally tends to prescribe psychiatric drugs. The medication approach starts typically with trial and error and dosage adjustment. The clinical trial data for most of the medication show a higher degree of side effects compared with placebo. The efficacy rate varies from person to person. The Food and Drug Administration generally approves these drugs based on the 200-300 patient clinical data based on risk and benefits approach. Most of the medications carry black box warnings on their package inserts. It is estimated that medication does not work for 40% of patients. Still, they suffer significant side effects for months and years before being classified as treatment-resistant or try other alternate approaches. The side effects of these medications may include serious conditions include deaths. For example: According to the FAERS (FDA's Adverse Event Reporting System), as of Dec. 31, 2018, there are 52,825 serious adverse events and 6,018 deaths related to a commonly prescribed drug (Risperidone) as an antipsychotic medication. As per the FDA, these are only reported events, and the majority of cases are not reported to the agency.

The current devices or systems rely on a clinical interview or genetic or imaging or neurological test to develop a treatment plan. Proper treatment is based on an appropriate diagnosis. An unreliable diagnosis generates unreliable treatment, which may result in significant side effects, including deaths without any improvement.

Currently, there is not a reliable diagnostic approach to identify mental disorder, and treatment is based on trial and error methods with significant side effects related to medication. Presently, there is no specific diagnostic approach based on multifactorial scoring based on genetic, neurological, and clinical data, utilizing machine learning and AI modeling. There have been patent filings for AI and the machine learning for health assessment, but there has been no known invention to combine the genomic, imaging, neurological, and laboratory data to generate a score based on comparing with phenotype and statistical pipeline.

### SUMMARY

This invention collects, correlate, process, and analyze inputs from genomic, imaging, and neurological data report to develop a multifactorial score based on the machine learning based on the statistical modeling based on the decision trees and naïve bays to improve sensitivity or specificity of diagnosis. This invention also supports a targeted treatment assessment using genetic, and pharmacogenetics filters and links any clinical and postmarked data.

This software and system invention links and consolidates inputs from sources such as genetic reports, imaging results, neurological tests, and clinical information and compare the data from current research to develop score using machine learning algorithms and data analysis techniques including linear and logistics regression, decision trees, Naive Bayes, support vector machines, and ensemble methods. This scoring and data analysis and overlay method helps to improve the sensitivity and specificity of complex illnesses such as mental disorders. This software and system also link treatment assessment plan to continuously learn and update diagnosis or treatment based on the new incoming data and current research using statistical modeling techniques.

### BRIEF DESCRIPTION OF THE DRAWINGS

FIG. 1: is the user set-up and data collection flow chart. This is used to set up a user/organizational account

FIG. 2: is the genetic data compilation and analysis process. This is used to collect, upload, and analysis the genomic data and generate the genomic report.

FIG. 3: is the imaging data compilation and analysis process. This is used to collect, upload, and analysis imaging data and imaging analysis report.

FIG. 4: is the multifactorial data analysis and reporting process. This is used to develop a comprehensive multifactorial diagnostic report.

FIG. 5: is the treatment assessment based on the diagnostic report. This is used to develop a treatment assessment report based on diagnostic reports and pharmacogenetic filters, and current drug databases.

### DETAILED DESCRIPTION

There is no reliable diagnostic and treatment approach to treat mental illness as it highly varies from the person to person and provider to provider. This is primarily due to the lack of a proper diagnostic approach. These diagnoses and treatments are generally based upon one-one meetings with a therapist or psychiatrist or a combination of both psychotherapy and psychiatric medication. Psychotherapy may include evidence based-techniques such as CBT and DBT. The other approaches may include meditation, light therapy, yoga, etc. Therapist are trained psychologist who generally start with psychotherapy and other evidence techniques whereas psychiatrist generally tends to prescribe psychiatric drugs. The medication approach starts typically with trial and error and dosage adjustment. The clinical trial data for most of the medication show a higher degree of side effects compared with placebo. The efficacy rate varies from person to person. The Food and Drug Administration generally approves these drugs based on the 200-300 patient clinical data based on risk and benefits approach. Most of the medications carry black box warnings on their package inserts. It is estimated that medication does not work for 40% of patients, but they suffer significant side effects for months and years before being classified as treatment-resistant or try other alternate approaches. The side effects of these medications may include serious conditions include deaths. For example: According to the FAERS (FDA's Adverse Event Reporting System), as of Dec. 31, 2018, there are 52,825 serious adverse events and 6,018 deaths related a commonly prescribed drug as an antipsychotic medication. As per the FDA, these are only reported events, and the majority of cases are not reported to the agency.

The invention is intended to improve the sensitivity and specificity of mental illness diagnosis using a multifactorial input approach. It is well understood that mental illness disorders are a combination of genetic and environmental factors. There is significant research in the area to identify genes involved in the mental disorder, including efforts by PsychCODE and Brain Initiative programs. It is also understood that this not a single gene but polygenic as a combination of genetic factors could impact the mental illness conditions. Some tests provide genetic information for specific genes, but there is no invention in the area to combine the genetic information from several genes and to correlate the genetic information with a phenotype of imaging, neuropsychic, and clinical information using machine learning techniques and statistical modeling to assess mental illness.

The claimed invention differs from what currently exists. The invention is an improvement over the existing methods as it combines the information from genetic, imaging, and neurological tests to develop a score based on the statistical modeling using the current and available clinical research to improve the sensitivity and specificity of diagnosis of mental illness. The invention also links with available drugs and filtering using genetic and pharmacogenetics profiles for targeted treatment if medication is required.

The invention is an improvement over the existing methods as it combines the information from genetic, imaging, and neurological tests to develop a score based on the statistical modeling using the current and available clinical research to improve the sensitivity and specificity of diagnosis of mental illness. The invention also links with known drugs and filtering using genetic and pharmacogenetics profile for targeted treatment if medication is required

The current systems are based silo approach and static in nature compared to closed-loop dynamic nature. These are either focused on genetics or imaging or just clinical interviews. Genotype, if available, is not linked with a phenotype such as imaging or neurological reports using machine learning algorithms based on statistical techniques to improve sensitivity and specificity of diagnosis. Additionally, these current diagnostic systems do not link with drug and therapeutic drug registries using filters to provide genetic pharmacogenetics screening to avoid extensive side effects if patients are placed on any specific medication and drug regimen.

This software and system invention links and consolidates inputs from sources such as genetic reports, imaging results, neurological tests, and clinical information and compare the data from current research to develop score using machine learning algorithms and data analysis techniques including linear and logistics regression, decision trees, Naive Bayes, support vector machines, and ensemble methods. This scoring and data analysis, and overlay method help to improve the sensitivity and specificity of complex illnesses such as mental disorders. This software and system also link treatment assessment to continuously learn and update diagnosis or treatment based on the new incoming data and current research using statistical modeling techniques.

### Components

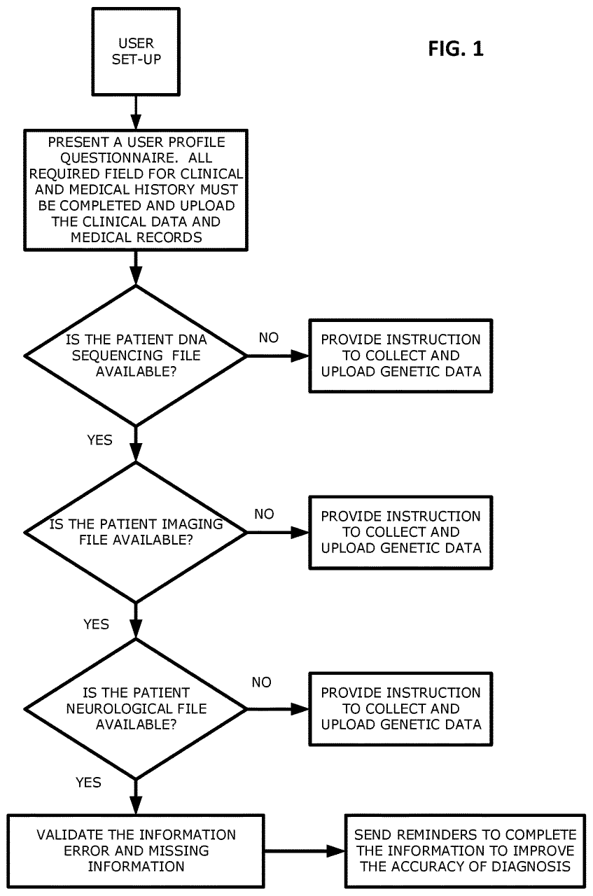

### 1. FIG. 1: User Set-Up and Data Collection

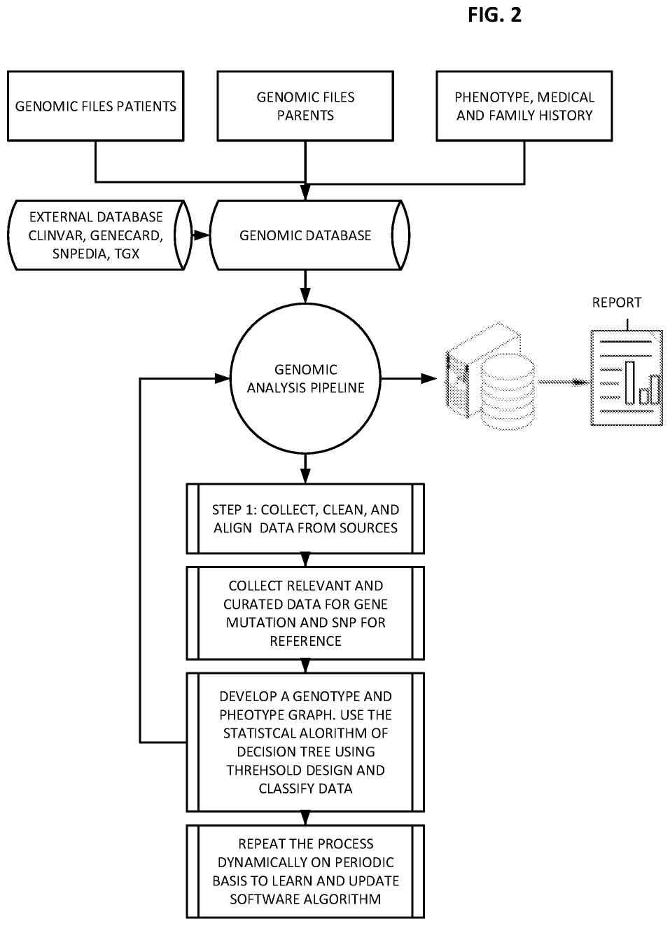

### 2. FIG. 2: Genetic Data Analysis

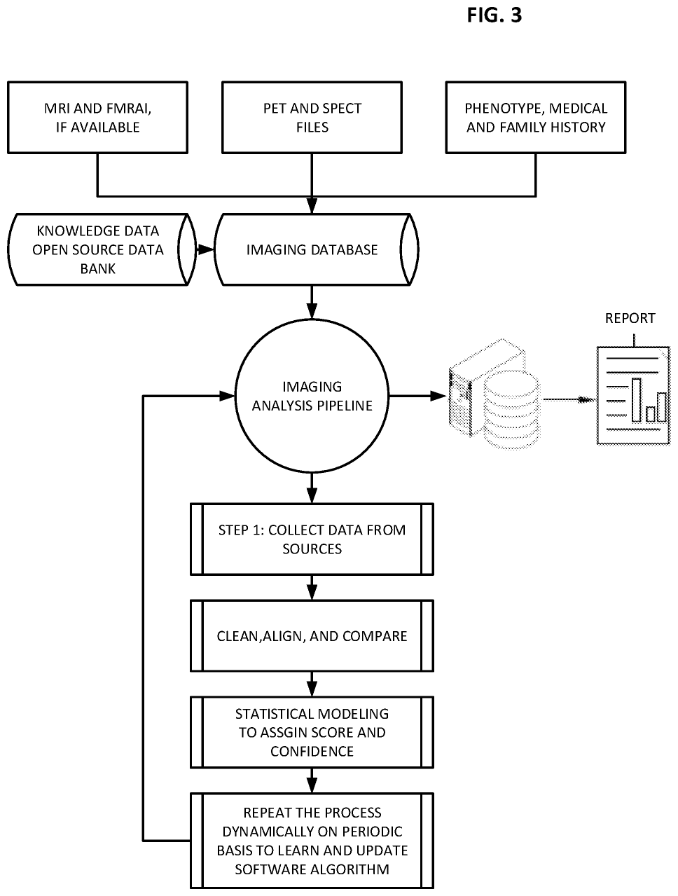

### 3. FIG. 3: Imaging Data Analysis

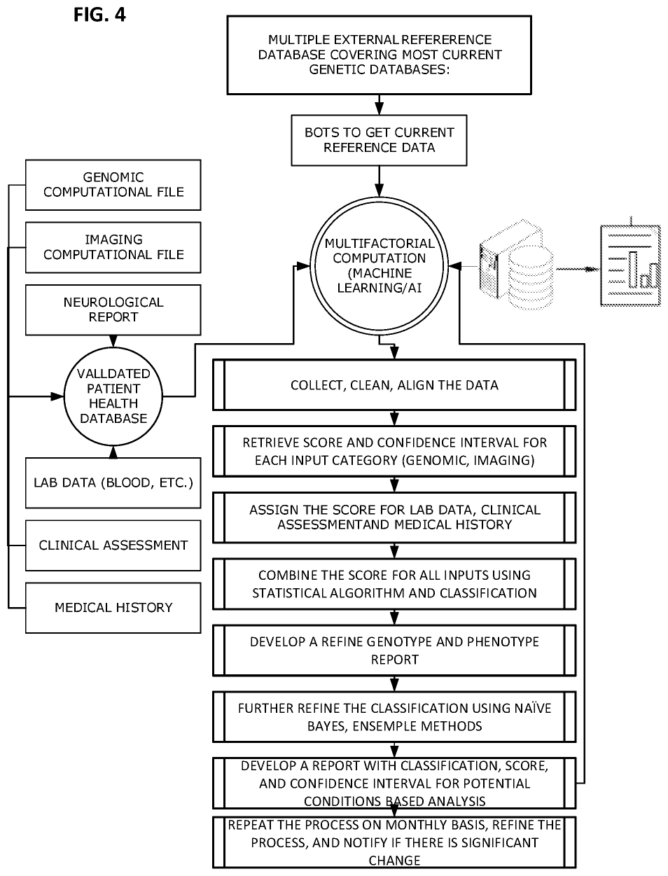

### 4. FIG. 4: Multifactorial Data Analysis

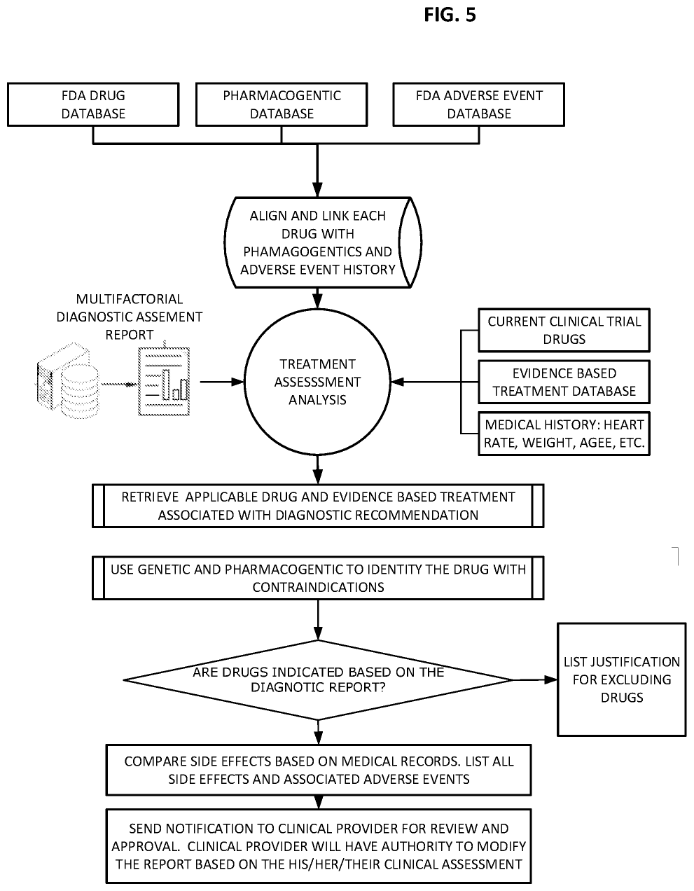

### 5. FIG. 5: Treatment Assessment

### Relationship Between The Components

Users (Patients and Providers/Doctors/Therapists) will access a desktop computer or mobile devices to access the cloud-based system through a webpage or apps. A user set-up and data collection process in FIG. 1 is initiated to provide user information either by responding to prompts and direct uploading of data such as DNA and medical files. Each patient have a unique and secure profile that will serve as primary identity, and all of the related information is collected in applicable relational tables/databases. Providers will have unique user profiles based on their roles and permissions. The graphical user interface (GUI) will link front-end software with the back-end server side to receive, process, analyze and generate scoring results based on statistical techniques as a part of software algorithms. After the user set-up process is completed, as listed in Exhibit A. A process of the multifactorial diagnostic will be initiated as provided in FIG. 4. If genomic data is uploaded, then the system will initiate the genetic reporting module in FIG. 2. If imaging data is uploaded, the system will initiate the imaging data analysis module in FIG. 3. Once genetic and imaging data analysis has been completed, the system will return to Multifactorial Diagnostic Module in FIG. 4 to analyze and report the multifactorial diagnostic process using the algorithm based on the machine learning to develop score and assign probabilities based on the statistical model.

Once the multifactorial diagnostic report is finalized using FIG. 4, then if the option of Treatment Assessment module is selected, the system will trigger the treatment module in FIG. 5 to generate a treatment assessment plan. The system will pull any associated approved drugs and/or therapy information using internal and external databases based on the multifactorial diagnostic analysis. The system will use genetic and pharmacogenetics filters to identify and flag drugs that may cause side effects and are ineffective based on the genetic profile. The system also filters based on vital signs such as high or low heart rate or blood pressure that may contribute to additional adverse events. The system will pull adverse event data from the FDA database (FAERS). The system will also link any alternate medical treatment such as TMS and other evidence-based treatment such as CBT and DBT.

The system will generate daily (if necessary) and weekly reports. The system is dynamic as it will use the bot approach to continuously update the information in the multifactorial diagnostic and treatment module. The system will generate a notification if there any changes necessary based on the updated diagnostic and treatment plan based on the most research or data or increase in adverse events or any warning from the FDA or other institutions. This ensures that the diagnostic and treatment plan is updated based on real-time monitoring and updated research.

### How The Invention Works

The system and software comprised on three primary and interlinked modules:

1. User Set-Up and Data Collection (FIG. 1)

2. Multifactorial Diagnosis Module (FIG. 2, FIG. 3, and FIG. 4)

3. Treatment Assessment Module (FIG. 5)

The multifactorial diagnosis module has two sub-modules: Genetic Data Analysis Module (FIG. 2) and Imaging Data Analysis Module (FIG. 3). The role of the User-Set-Up and Data Collection (FIG. 1) module is the create patient and provider profiles with specific access with permissions and specific roles. This module (FIG. 1) is also used to upload patient data and files for processing.

The role of the multifactorial diagnosis module (FIG. 4) is collecting and correlating multiple data inputs covering genomic, imaging, neurological, clinical, medical, physical, nutritional, etc. Genetic Reporting Module (FIG. 2) and Imaging Module (FIG. 3) are sub-modules for the multifactorial diagnostic module (FIG. 4). Once patient data is collected and binned, then the system extract and collects internal and external relevant data based on the current research for comparison. Once the patient data and internal/external data is organized and binned, then the system uses the statistical modeling based decision tree, naive Bayes, support vector machines, and ensemble methods to classify the data and develop recommendation with applicable probabilities. A report is generated based on the data analysis and presented to review and approval. The role of the treatment assessment module (FIG. 5) is to collect relevant treatment plan information based on the diagnostic recommendation. The systems use the genetic and pharmacogenetics filters to identify and flag treatment that may be ineffective based on the genetic and pharmacogenetics information. The system then adds any FDA adverse events data to the remaining treatment options make providers aware of these adverse events and warnings. This module (A comprehensive treatment report is developed for review and approval by the treatment team.

The process starts with user set-up as shown in (FIG. 1: User Set-Up and Data Collection.) After user set-up has been completed, verified, and validated, then the system will initiate the multifactorial diagnostic module as provided in (FIG. 2: Multifactorial Data Analysis). The multifactorial diagnostic module will trigger a genetic report module (FIG. 2) if genomic data is provided for patients and parents. The genetic data analysis (FIG. 2) is based on statistical modeling to compare genotype with phenotype and statistical modeling techniques such as Naive Bayes, Support Vector Machines, and Ensemble Methods to classify the information and data. If imagining and neurological data are provided, then the diagnostic module will also initiate the imaging module (FIG. 3). Once all required diagnostic inputs are collected, then the multifactorial diagnostic module (FIG. 4) will process, analyze, and generate a report using a statistical model based on the machine learning process.

Once the multifactorial diagnostic process has been completed, then the diagnostic report will become an input to the treatment assessment module (FIG. 5). The module will collect and retrieve relevant treatment recommendations using an internal database and also retrieve external data. The treatment assessment module (FIG. 5) will use filters such as genetic and pharmacogenetics filter to flag treatments that may be ineffective and cause adverse events.

The system must link multiple inputs covering genotype and phenotypes related to mental illness. The system must be able to compare patient data with any applicable reference data for genomics and imaging data if available. This system must be able to link and retrieve drugs and treatments from drug registries. The system must be able to receive and process inputs for the monitoring phase and create a notification. The system is dynamic in nature to continuously monitor and update the knowledge database based on the machine learning algorithm. The system must be able to perform the tasks listed in FIG. 1, FIG. 2, FIG. 3, FIG. 4, and FIG. 5.

### Operations

### Step 1: User Set-Up and Data Collection (FIG. 1)

Provider and Administrative Profiles are developed as a part of mental health facilities in the cloud-based system using the web interface. A user with administrative rights will set up a user account and specific rights. Once a specific user account is established, then the user will provide the applicable information and upload the data files for the back-end system to analyze.

### Step 2: Genomic Data Analysis (FIG. 2)

The system will use the data and information uploaded to perform genomic data analysis. The genomic data analysis will compile patient genomic data, parents genomic data (if available) with medical history. This will use the available reference data linked with a mental health condition and align the patient genomic with parent genomic data with genomic reference data to identify matches. The system will use a comparison and software algorithm to generate a report with potential conditions with score and confidence interval. This report will become one of the inputs for the multifactorial diagnostic process in FIG. 4.

### Step 3: Imaging Data Analysis (FIG. 3)

If imaging data is uploaded using FIG. 1, the system will use FIG. 3 (Imaging Data Analysis) to develop an imaging analysis report as a part of a diagnosis. The imaging data analysis uses imaging files for MRI, PET, SPECT and retrieves linked mental illness modalities associated with imaging files. The statistical analysis report will compare phenotype and develop a list of potential conditions with score and confidence interval. The assigned score will be an input to multifactorial data analysis (FIG. 4).

### Step 4: Multifactorial Diagnostic Data Analysis (FIG. 4)

The system will use the data and information uploaded to perform multifactorial diagnostic assessments. Genomic and imaging data files are uploaded, the system will use the score from genomic and imaging data analysis and combine the information from neurological, medical, and lab reports to develop a multifactorial diagnostic report. The multifactorial diagnostic process combines inputs from various sources. The diagnosis will be based on the modeling of multiple inputs covering genetic, medical, neurological, clinical, imaging, etc. by assigning weight to each input and creates a multifactorial based comprehensive score for each potential condition with confidence interval. The statistical modeling and machine learning approach improves the sensitivity and specificity of diagnosis.

### Step 5: Treatment Assessment (FIG. 5)

The system will compile a treatment recommendation on the based diagnostic recommendation using the approach based on multifactorial diagnostic analysis in FIG. 4. For example, if a diagnostic report recommends a bipolar condition with high probability based on the score and confidence interval, then the system will fetch for all available drugs and evidence-based treatments for this condition, then use the genetic and pharmacogenetics filters to flag drugs that may be ineffective or cause adverse events.

### CONCLUSION

Mental illness is a complex condition, and this could be related to multiple factors, including genetic (germline or somatic), physical (changes in brain and other physical medical conditions), environment, etc. This invention is an improvement over the existing methods. It combines the information from genetic, imaging, and neurological tests to develop a score based on the statistical modeling using the current and available clinical research to improve the sensitivity and specificity of diagnosis of mental illness. The invention also links with available drugs and filtering using genetic and pharmacogenetics profiles for targeted treatment if medication is required.

I claim:

1. A diagnostic and treatment assessment system for mental illness based on collecting and analyzing using multifactorial design comprising:
a. Compiling and consolidating genomics, imaging, medical, and clinical data to align with reference data;
b. comparing the genomic, imaging, and clinical data with phenotype;
c. statistical modeling of decision tree with a threshold to classify the data;
d. refining the statistical modeling based on naïve bays and support vector; and
e. assigning a multifactorial score based on the probability for diagnostic reports.

2. A treatment assessment plan based on the multifactorial diagnostic assessment and using pharmacogenetic and risk factors to refine the treatment assessment.
